(19) United States
(12) Patent Application Publication
(10) Pub. No.: US 2022/0249393 A1
(43) Pub. Date: Aug. 11, 2022

(54) COMPOSITION OF ACTIVE INGREDIENT LOADED EDIBLE INK AND METHODS OF MAKING SUITABLE SUBSTRATES FOR ACTIVE INGREDIENT PRINTING ON ORODISPERSIBLE FILMS

(71) Applicant: ZIM LABORATORIES LIMITED, Nagpur (IN)

(72) Inventors: Anwar DAUD, Nagpur (IN); Nidhi SAPKAL, Nagpur (IN); Vaishali KILOR, Nagpur (IN); Minal BONDE, Nagpur (IN); Priya DULE, Nagpur (IN)

(73) Assignee: ZIM LABORATORIES LIMITED, Nagpur (IN)

(21) Appl. No.: 17/047,063

(22) PCT Filed: Apr. 11, 2019
(86) PCT No.: PCT/IN2019/050299

§ 371 Date: Oct. 12, 2020

(30) Foreign Application Priority Data
Apr. 13, 2018 (IN) 201821014188

(51) Int. Cl. A61K 9/70; A61K 9/00; A61K 47/38; A61K 47/10; A61K 47/26; A61K 47/02; A61K 47/44; A61K 47/22; A61K 47/34

(52) U.S. Cl. CPC A61K 9/7007; A61K 9/006; A61K 47/38; A61K 47/10; A61K 47/34; A61K 47/02; A61K 47/44; A61K 47/22; A61K 47/26

(57) ABSTRACT

This invention discloses an orodispersible thin film for providing a substrate for printing of at least one Active ingredient on its surface that remains free from cavities after printing, method making the film, the substrate for the printing and a hydrophobic edible ink comprising at least one Active Ingredient for the printing. The Invention includes all films having orodispersibility property; including rectal, vaginal, ocular film and any other film meant for oral or transmucosal delivery. One or more of ingredient/s that make the substrate of this invention act as adsorbents and impart/s required roughness to the surface of the substrate. The ink does not crystallize on drying and remains stable at least for six months at 40° C. and 75% RH; and is printable using a Continuous Inkjet printer.

### FIELD OF THE INVENTION

The present investigation relates to compositions for the Active Ingredient-containing printing ink as well as methods for making substrates suitable for printing to make Active Ingredient printed orodispersible thin films for oral and transmucosal delivery.

### BACKGROUND OF THE INVENTION

Thin films are the pharmaceutical dosage forms that can be administered for transmucosal delivery through oral, sublingual, buccal route for both systemic and local action. Oral thin films are very much popular as an Active Ingredient delivery system as these quickly dissolve when kept on the tongue. This oral dosage form is very much convenient for geriatric and pediatric patients since it does not require water for swallowing and can be administered to uncooperative and unconscious patients as well. There is no need for water for administering this dosage form. More surface area results in better solubility and improved dissolution.

There are various methods of manufacturing of orodispersible films like solvent casting, hotmelt extrusion, semisolid casting, solid dispersion extrusion, and rolling. Among several techniques of film manufacturing, solvent casting is preferable and widely used method mainly due to the simple and continuous manufacturing process and low cost of processing.

Despite the apparent simplicity, it is potentially challenging to guarantee thickness and content uniformity when casting films over several meters (JanBen et al., 2013). Highly sophisticated controls are required on the casting equipment to meet these challenges that make the film forming process complicated and expensive. Furthermore, cutting cast films with knives or punches can create additional dosage error and unacceptable material waste (JanBen et al., 2013). This can be a serious drawback of this technology for the preparation of oral thin films of controlled substances as well as potent active ingredients.

Further, in the solvent casting of Active Ingredient loaded films, mechanical properties of films may change depending upon the amount and chemical nature of the actives. Presence of a large amount of aqueous solvent in casting dispersion affects the stability of many actives, which are prone to hydrolytic degradation.

Solvent casted films utilize heat for drying of the films which may affect the stability of thermolabile Active Ingredients.

In the case of rapidly dissolving oro-dispersible films, the main challenge is to produce films with a rapid disintegration/dissolution time without compromising the mechanical properties of the films. Some actives have the tendency to crystallize due to super-saturation during drying phase which could potentially change the mechanical properties of the film, alter the dissolution rate of the Active Ingredient, change the mouth feel and taste of the Active Ingredient and subsequently alter the in-vivo fate of the Active Ingredient.

Actives prone to oxidation have to be protected well since stirring of polymeric dispersion creates aeration in the mixing vessel.

In this context, manufacturing rapidly dissolving oro-dispersible films by printing actives onto placebo substrates can overcome these limitations. The Active Ingredient printing process also increases the production yield and product quality and the number of actives can be delivered using thin film dosage form by printing more than one Active Ingredient on one placebo film. This technique is especially useful for those Active ingredients which cannot be developed into thin film formulations. Printing technologies specifically digital inkjet printing or pneumatic based extrusion printing, offer possibilities in the production of individualized medicines. Inkjet printing is classified as Continuous Inkjet printing and Drop-on-Demand printing. Drop-on-Demand technique is further classified as Thermal Inkjet printing as well as Piezoelectric Inkjet (PIJ) printing. The main advantage of inkjet printing includes the ability to dispense uniform droplets in the pico-litre range with a high degree of accuracy to allow dose personalization. Inkjet printing is a recently explored method of manufacture, which involves the uses of a substrate or matrix for printing and the use of an Active ingredient-loaded liquid phase.

The challenge of printing of actives onto thin film is to deposit the accurate dosage in the precise and specific surface area of thin films without compromising other properties of the film like physical appearance, mechanical strength, disintegration time, release rate, etc.

Orodispersible films (ODF's) are the films that would disperse when delivered in oral cavity. For the purpose of these claims and this specification the term “Orodispersible” is used to define the property of the films, intended to be covered within the scope of these claims and this specification, to the extent that these films would disperse if kept in oral cavity; but need not be intended to be restricted to an oral film only; it also covers all film dosage forms having properties same as orodisperible films and cover, for example also the films of types, sublingual, vaginal film, ocular film and the like i.e. those films which would disperse when placed in oral cavity.

Melendez et al. (2008) reported printing of prednisolone using thermal inkjet (TIJ) printing. Printing solution was prepared by dissolving the API in an ethanol-water-glycerol solvent system. The substrate was polytetrafluoroethylene (PTFE)-coated fiberglass film. This substrate is not edible therefore not useful for the delivery of Active ingredients. The ink composition described in this report contains components that form cavities when printed on the edible/pharmaceutical substrate.

N. Genina et al. (2013) evaluated the applicability of the different model substrates, namely orodispersible films (ODFs), porous copy paper sheets, and water impermeable transparency films (TFs) in preparation of the inkjet-printed drug-delivery systems. Printing of Rasagiline mesylate ink (containing Propylene Glycol:water in 30:70 volume %) was carried out on Oral dissolving Film (containing 14% HPMC, 4.7% cross-povidone, 4.2% glycerol and 77.1% water). It was observed that cavities are formed on ODF substrate during printing since the ink contains water and hydrophilic material propylene glycol. Porous copy paper as well as TFs films could retain the ink. Both porous copy paper and TFs are not appropriate substrates for the purpose of orodispersible films.

Sandler et al. (2011) reported printing of an API solution on a porous substrate to study the potential of the approach for incorporation of different API formulations into these types of structures. They used a PH printer to deposit three different API formulations (theophylline, acetaminophen, and caffeine) using three types of substrates uncoated paper, pigment-coated paper, and polyethylene terephthalate (PET) films. The ink solutions containing the drug substances were dissolved in Propylene Glycol:purified water (30:70 vol %). However, the substrates used in this work are not acceptable for the purpose of orally dissolving films.

Pardeike et al. (2011) introduced a concept of printing of API nanoparticles onto edible sheets by inkjet printing. Nano suspension of Folic acid prepared by suspending 10% (w/w) folic acid in an aqueous 3% (w/w) Tween 20 solution and was used as an ink. The ink formulation disclosed in this work contains water and hydrophillic surfactant which is bound to affect the critical quality attributes of the printed films for oral/transmucosal delivery. It does not disclose composition of substrate in order to assess its suitability as orodispersible film.

Genina et al., 2013 reported printing of caffeine and loperamide hydrochloride on three different substrates: edible icing sheets, hydroxypropyl cellulose (HPC) films and PET films. A water-based ink was chosen to prepare printable formulation for caffeine by dissolving API powder in 30:70 (vol %) mixture of PG and water. The loperamide ink was prepared by dissolving API crystals in 40:60 (vol %) mixture of PG and ethanol. The ink compositions in this paper are highly hydrophilic and are capable of dissolving the edible substrates i.e. icing sheets and HPC films. Such ink compositions will not yield printed oral/transmucosal films with desirable attributes.

Raijada et al. (2013) formulated printable dosage forms for piroxicam. Different ratios of PEG:ethanol formulations (30:70, 40:60, 50:50) were used for inkjet printing on edible paper using PH and impression printing (flexography). In our experience, ink compositions mentioned in this work form cavities during printing on the substrates suitable for the purpose of orodispersible films.

Vuddanda et al. formulated warfarin printed films by using Thermal InkJet printer where the substrate (paper) rolling mechanism of the printer was replaced by printing onto a stationary stage. Free film substrates were composed of hydroxypropyl methylcellulose (HPMC) (20% w/w) and glycerol (3% w/w). Aqueous solution of warfarin was used as an ink in the present work, which pose a problem of cavity formation on the hydrophilic substrate prepared using glycerol and HPMC.

Buanz et. al. (2011) evaluated the use of thermal inkjet printing as a method for dosing drugs onto oral films. Aqueous solution of Salbutamol sulphate (3% w/v) containing glycerine (10% v/v) was used as an ink for printing onto a commercial potato starch film. The drug printed films disclosed in this literature does not yield orodispersible films that can withstand routine pressures of manufacturing while retaining all the critical quality attributes.

Buanz, et al. (2014), reported Inkjet printing to prepare oral films. The substrate was composed of Polyvinyl alcohol and sodium carboxymenthyl cellulose (NaCMC) at 1:1 ratio with 24% w/v glycerol. Printing ink composed of Clonidine (50 mg/ml) prepared in 20% v/v methanol in water with 10% v/v glycerol. The substrate used for printing in this work is hydrophilic in nature and ink contains large amount of water and glycerol. Such a composition of ink is not capable of printing on the reported substrate while retaining all the desired critical quality attributes of the substrate after printing.

Planchette, et al. (2015), studied printing of Sodium picosulphate in three ink compositions: (i) an aqueous API solution, (ii) placebo nano suspensions and (iii) PEG solutions. The printing was done onto medical-grade oral films by using both PH and solenoid valve-based inkjet technologies. As medical grade oral films are designed to disintegrate rapidly in oral cavity those are composed of hydrophilic film forming polymers. When aqueous API solutions are printed on theses films it is difficult to retain the desired attributes of oral films.

The challenge of printing of actives onto thin film is to deposit the accurate dosage in the precise and specific surface area of thin films without compromising properties of film including, without limitation, mucoadhesion, physical appearance, mechanical/tensile strength, disintegration time, release rate, folding endurance, dissolution rate, and Active Ingredient content. All studies reported above include the use of substrates such as icing sheets, starch sheets, medical grade oral films, Polyethylene terephthalate films (PET) and pigment coated paper; potato starch sheets, All these materials either lack mechanical strength required for pharmaceutical processing or are not pharmaceutically acceptable material. Each one of these substrates has drawbacks. The icing sheet is extremely delicate and cannot be handled in routine manufacturing. Starch sheets have very less mechanical strength. PET films are not water soluble and are also not edible. The pigment coated paper is composed of water insoluble material, and is not edible, hence not useful for making oro-dispersible films.

Films made from hydrophilic polymers show unsuitability for printing by water based inks or inks containing any hydrophilic ingredient as it dissolves and forms cavities/depressions after deposition of ink during printing. It was observed that none of the above mentioned substrates have all the desired Critical Quality Attributes (CQAs) required to act as an orodispersible thin film for transmucosal delivery of an Active ingredient. The CQAs of the films comprise folding endurance of minimum 8, disintegration time of minimum 30 sec and minimum mechanical/tensile strength of 9 kg/mm2 for oral and sublingual film additionally mucoadhesivity for buccal film.

Properties of Active ingredient containing ink are also required to be modified in order to make the process realistic while maintaining the stability of Active Ingredient loaded in it. Ink formulations used by earlier researchers mentioned the use of water-based ink, which is practically not possible while printing substrate containing hydrophilic film forming material.

As both the components contain large amount of water it is very much likely to form cavities in the substrate due to the dissolution of hydrophilic components of the substrate in water based ink. Therefore, it is required to formulate Active Ingredient loaded ink which will not lead to cavity formation after printing while retaining other attributes essential for the good quality printing process. Thus, there remains a need for suitable ink composition as well as substrate composition suitable for Active Ingredient printing using printing technique which is capable of retaining the desired quality attributes of the Active Ingredient printed film like mechanical strength, Active Ingredient stability and fast disintegration as well as or mucoadhesion and other relevant properties.

For the purpose of this specification, “Active Ingredient” includes Active Ingredients as well as non-Active Ingredient ingredients that are useful for healthcare.

For that matter there is too little knowledge on technology of printing Active Ingredients on orodispersible films and systematic investigation in this matter is needed.

### SUMMARY OF THE INVENTION

This invention discloses an orodispersible thin film for providing a substrate for printing of at least one Active ingredient on its surface that remains free from cavities after printing. For the purpose of these claims and this specification the term “Orodispersible” is used to define the property of the films an intended to be covered within the scope of these claims and this specification to the extent that these films would disperse if kept in oral cavity; but need not be intended to be an oral film only, it may cover films of the defined property but may be intended to be rectal film, vaginal film, ocular film and the like.

The orodispersible thin film of this invention is characterized by having folding endurance of minimum 8 times without breaking, disintegration time of minimum 10 seconds after coming in contact with water and minimum mechanical/tensile strength of 9 kg/mm².

The orodispersible thin film according to this invention is one selected from the group consisting of buccal, sub-lingual, oral, vaginal, rectal and ocular film and any other film meant for oral or transmucosal delivery.

The orodispersible thin film according to this invention has following characteristics:

a. is made from one or more film forming polymers comprising one or more of Hydroxypropylmethyl cellulose, Hydroxypropyl cellulose, Polyethylene Oxide, Pullulan, maltodextrin, sodium alginate, Gelatin, carrageenan, chitosan, Polyvinyl Pyrrolidone, Poly Vinyl Alcohol, Sodium carboxymethyl cellulose, and Modified starch and the like,

b. comprises one or more plasticizers comprising Polyethylene Glycol, propylene glycol, glycerin, sorbitol, triacetin, and mannitol and the like,

This invention also comprises a substrate for making an orodispersible thin film that has printing on its surface with at least one Active Ingredient for oral/transmucosal delivery. The substrate of this invention is characterized in that the said substrate has paper like surface roughness, porosity and absorptivity for the purpose of printing; and foldability and mechanical strength for the purpose of withstanding handling in course of its manufacturing, packaging, storage, transportation and use. One or more of ingredient/s that make the substrate of this invention are chosen to act as adsorbent/s and impart/s required roughness to the surface of the substrate.

The substrate according to this invention is made from ingredients selected from the group consisting of Microcrystalline Cellulose, gelatin, Polyethylene Glycol-2000, Polyethylene Glycol-4000, Polyethylene Glycol-6000, Titanium Dioxide, Kaolin, Kieselguhr, Bentonite and Neucilin. The selected ingredient is used in the range 5-10% w/w.

This invention also comprises a method for making a substrate for making an orodispersible thin film for providing a substrate for printing of at least one Active ingredient on its surface that remains free from cavities after printing. The method for comprising the steps of: (a) adding weighted quantity of film forming polymer, propylene glycol, glycerin, mentha oil, Simethicone and purified water was added in a Stainless Steel container, stirring to get a mixture, (b) adding one or a mixture of polymers that would provide paper like consistency to the mixture upon drying for the purpose of printing of the Active Ingredient, and homogenized to make a homogeneous dispersion, (d) casting the homogeneous dispersion in uniform thickness, and (e) drying to get a film. The method for making the substrate according by above method is selected one or more a polymer comprising Hydroxypropyl methyl cellulose, hydroxy propyl cellulose, pullulan, sodium alginate and polyethylene oxide, casting of the film is done using film casting machine and drying is done at 80° C. for 10 minutes. The substrate made by this method comprises mucoadhesive films.

This invention also discloses an ink for printing on an orodispersible film at least one Active ingredient without causing cavities after printing. The ink does not cause dissolution of the substrate during printing, The ink according to this invention is edible, dries fast after printing on a substrate, comprises surface tension, viscosity and contact angle required for adherence of ink to the substrate, does not crystallize on drying, remains stable at least for six months at 40° C. and 75% RH, comprises one or more viscosity modifiers comprising cellulosic polymers comprising Hydroxypropyl cellulose, ethyl cellulose, sodium alginate, Hydroxy methyl cellulose of Low viscosity chitosan, xanthin gum, pectin, carrageenan and antioxidants like Butylated Hydroxy Anisole, Butylated Hydroxy Tolune, Tocopherol; and conductivity imparter like sodium chloride or other salts appropriately equivalent to Sodium Chloride for the purpose of the ink composition, The ink of this invention also comprises one or more surfactant/s to solubilise the Active Ingredient, the surfactants being one or more comprising Tween 80, Tween 20, Span 80, Span 20, Brij, Poly Ethylene Glycols, pluronics, sorbitol esters, alkyl sulphates, alkyl ether sulphate, polyunsaturated fatty acid esters, polyoxyethylene glycol fatty acid ester, polyoxyethylene stearate and Vitamin E acetate, The ink also comprises excipients that are pharmaceutically and/or nutraceutically acceptable, The ink also comprises one or more Active Ingredients comprising drugs and Nature derived healthcare ingredients including nutraceutical ingredient.

The ink of this invention remains stable for at least six months at 40° C./75% RH. The ink of this invention comprises ethanol or hydroalcoholic mixture. The ink of this invention also comprises a solvent. The ink of this invention also comprises Butylated Hydroxy Toluene, Butylated Hydroxy Anisole, Tocopherol and other antioxidants. The ink also comprises surfactants. The ink also comprises cellulosic polymers as viscosity enhancers including, without limitation, Hydroxypropyl cellulose, ethyl cellulose, sodium alginate and Hydroxy methyl cellulose of Low viscosity. The ink of this invention dries fast when printed on the substrate. The ink of this invention is also printable on the substrate using a Continuous Inkjet printer.

### DETAILED DESCRIPTION OF THE INVENTION

The composition of Active Ingredient loaded printing ink of this invention is suitable for printing on placebo substrate or plain oral thin film in which no Active Ingredient has been incorporated. In general, Active Ingredients of diverse types and in particular Active Ingredients or APIs, from the BCS class I and II like Clonazepam, Diazepam, Diclofenac, Piroxicam, Theophylline, Salbutamol Sulfate, Cholecalciferol, Ketorolac tromethamine, Danazol, Albendazole, Danazol, Ketoconazole, Atovaquone, Nifedipine, Carbamazepine, Piroxicam, Phenoxymethylpenicillin-potassium, Glimepiride, Levofloxacin, Nimesulide, Griseofulvin, valacyclovir, gabapentin, furosemide, levodopa, metformin, and ranitidine HCl, amitriptyline hydrochloride, biperiden hydrochloride, chloroquine phosphate, chlorpheniramine maleate, chlorpromazine hydrochloride, clomiphene citrate, cloxacillin sodium, cyclophosphamide, doxycycline, ergotamine tartrate, fluconazole, indinavir sulfate, levamisole hydrochloride, levothyroxine sodium, mefloquine hydrochloride, nelfinavir mesylate, neostigmine bromide, phenytoin sodium, prednisolone, promethazine hydrochloride, proguanil hydrochloride, quinine sulfate, warfarin sodium, caffeine, metoprolol, propranolol, verapamil, valacyclovir, diltiazem, gabapentin, levodopa, and divalproex sodium etc.

One of the aspects of the invention relates to a composition of suitable substrate possessing characteristics similar to orodispersible thin films but having properties that make them a good substrate for printing, such as good absorptivity and sufficient mechanical strength.

The present invention comprises a rapidly dissolving placebo polymeric film substrate containing water-soluble polymers prepared by a solvent casting method for printing with an edible aqueous ink containing or without an Active Ingredient. The active ingredient may be a Active Ingredient or other than a Active Ingredient. In one embodiment, the placebo polymeric film is made from hydrophilic polymers. In a further embodiment, the placebo polymeric film made from hydrophilic polymers is prepared by a solvent casting method. For the purpose of this specification a “Placebo polymeric film” covers within its scope rapidly dissolving solving orodispersible polymeric film that is cast for the purpose of providing a thin film substrate for printing of an Active Ingredient on its surface by an edible ink comprising the Active Ingredient. The Active Ingredient may be a pharmaceutical, Nutraceutical, cosmeceutical or any other healthcare ingredient.

In another aspect, the present invention comprises an Active Ingredient composition of Active Ingredient containing non-hydrophilic edible ink that is suitable for printing on substrate of the invented composition which yields the final product having paper-like properties and is pharmaceutically acceptable or edible. In case of unstable Active Ingredients, the ink is able to retain the stability of active ingredient in the ink formulation as well as in the printed form. The paper-like properties comprise foldability, absorptive properties, flexibility, slightly rough and porous surface; and despite these properties, the prepared, the films are edible and fast dissolving and possess physical and chemical properties as required by pharmaceutical or edible film dosage forms. In another embodiment the present invention comprises formulation or composition of Active Ingredient-containing ink solution ready for printing on placebo substrate (thin film) using Ink-Jet printer/pneumatic based extrusion printer without the formation of cavities or depression in the film. The resulting deposit has good adherence with the substrate and give stable films. After printing, the film the film can be cut into the desired size containing an accurate amount of dose per unit. The printing ink formulated in this invention is fast drying and has a suitable viscosity and surface tension required for suitable Active Ingredient printing on placebo substrate (film).

This invention also comprises a process of printing active constituent solutions on placebo films wherein the Actives may be heat sensitive, prone to hydrolysis.

This invention also comprises a process of printing active constituent solutions on placebo films wherein the Actives may be heat sensitive, prone to hydrolysis, potent, controlled substances or may be fixed dose compositions.

The active ingredient loaded ink, upon deposition, may control the release of an active agent by choosing the appropriate polymers or other means of controlling the release.

The active ingredient loaded ink of the present invention results in the Active Ingredient printed film with essential attributes of orodispersible thin films such as disintegration time, solubility, stability, physico-mechanical properties, etc.

The active ingredient may, for example, be medicinal, nutraceutical, dietary additive, cosmeceutical, colorant and a diagnostic. This is especially suitable for pharmaceutical, nutraceutical and dietary additives that might not be suitable for converting into thin films using solvent casting or hot melt extrusion process. Active ingredients which are unstable at casting temperature, potent Active Ingredients as well as controlled substances can be suitable Active Ingredient candidates for printing on the oral thin films.

This invention comprises a composition a of Active Ingredient loaded ink as well as a film substrate required in the process of preparation of Active Ingredient printed orodispersible thin films. Active Ingredient-loaded ink of this invention forms drops which dry fast after they are applied to a substrate during printing. The ink of this invention comprises optimum surface tension, viscosity and contact angle required for adherence of ink to the to the substrate. Surface tension was measured using stalagmometer where water was used as a reference standard. For the measurement of viscosity Ostwald's viscometer was used. The contact angle of the ink was measured by dropping ink on the substrate and the angle formed by the drop was observed with the help of magnifying glass using a protractor. Ink dries fast within 5 to 10 seconds at 25-30° C. after deposition.

Active Ingredients loaded in the Ink composition of this invention do not crystallize on drying. Active Ingredients loaded in the printing ink remains stable for six months at 40° C. and 75% RH.

The ink composition of the present invention contains viscosity modifiers from the category of cellulosic polymers like HPC, sodium alginate, chitosan, xanthan gum, Guar gum, pectin, carrageenan, methyl cellulose, NaCMC, carbopol, and ethyl cellulose. Ink of this invention contains surfactants like polysorbate 80, sorbitan monooleate, Polyethylene glycol, Vitamin E acetate to solubilise the Active Ingredient. Ink may also contain suitable antioxidants and conductivity enhancers if required. The ink may contain suitable colors, flavors, sweeteners, taste masking agents to improve the aesthetic appeal of the printed orodispersible films.

The present invention comprises a composition of a film substrate suitable for Active Ingredient printing at the same time retaining the required critical quality attributes of the film dosage form. For rapidly dissolving films. The critical attributes are folding endurance, mechanical strength, absence of cavity formation and fast disintegration. Additionally for mucoadhesive films bioadhesion is also required.

In another embodiment, the substrate is capable of holding the required dose of the Active Ingredient without any alteration in the original properties like mechanical strength, disintegration, folding endurance, bioadhesion etc.

In one more embodiment the said substrate has paper like appearance which is the requisite for Active Ingredient printing i.e. surface roughness, porosity, foldability, mechanical strength, absorptivity etc.

In another embodiment the substrate can be used for oral, buccal, sublingual, rectal or vaginal administration.

In one more embodiment the substrate is used for printing two incompatible Active Ingredients on the substrate surface simultaneously or in stepwise manner.

In another embodiment of the present invention comprises of composition of the substrate wherein printing can be done in various patterns like dots, lines, logo, letters or complete surface of the substrate can be printed.

In another embodiment of the present invention, ink containing nano suspension of sparingly soluble Active Ingredient can be printed on the substrate.

The Active Ingredient printed substrate or a film of the present investigation dissolves quickly as soon as placed on the tongue and does not require water for swallowing.

In another embodiment of the present invention the printed film adheres to the buccal/rectal/vaginal/sublingual mucosa for transmucosal delivery of Active Ingredient. In another embodiment of the present invention, the excipients used for substrate and ink are from GRAS category and therefore the substrate and ink can be used for printing pharmaceuticals.

In another embodiment, films or substrate of the present invention, after printing can be cut in to desired size containing an accurate amount of dose per piece which is desirable for accurate delivery of dosage.

In another embodiment the substrate in the said invention after Active Ingredient printing can be cut in to desired size, coiled and filled into the hard gelatin capsules for oral administration where printed Active Ingredient can be absorbed through gastrointestinal tract.

In one more embodiment maximum area of the substrate i.e. 90% can be utilized for Active Ingredient printing.

In another embodiment of the present invention the substrate contains Active Ingredient which is thermostable and on its surface thermolabile Active Ingredient loaded ink can be printed to form a fixed dose combination formulation.

Besides above mentioned embodiments, there may be several other embodiments that achieve good quality Active Ingredient printed thin films, including the ones which are obvious variants of above or equivalents of above, and all of them are included within the scope of disclosure of this specification. The non-limiting examples given below are only illustrative and do not limit the scope of means used for composition of suitable substrates and Active Ingredient loaded ink suitable for printing using inkjet printers based on varying principles.

### EXAMPLES

1. Composition of Active Ingredient Loaded Ink

1.1 Preparation of Vitamin D3 Printing Ink—(Ink A)

TABLE 1

| | | Amount in Percent |
|---|---|---|
| Sr. No | Ingredients | w/v |
| 1. | Vitamin D3 | 4.35 |
| 2. | BHA | 3.48 |
| 3. | BHT | 3.48 |
| 4. | Ethanol | 87 |
| 5. | Ethyl cellulose | 1.64 |
| 6. | Red iron oxide | 0.1 |

1.2 Preparation of Clonazepam Printing Ink—(Ink B)

TABLE 2

| | | Amount in Percent |
|---|---|---|
| Sr. No | Ingredients | w/v |
| 1. | Clonazepam | 5.35 |
| 2. | BHA | 2.48 |
| 3. | Sucralose | 1.43 |
| 4. | Ethanol | 87 |
| 5. | Ethyl cellulose | 1.74 |
| 6. | Strawberry flavor | 2.0 |

1.3 Preparation of Loperamide Printing Ink—(Ink C)

TABLE 3

| | | Amount in Percent |
|---|---|---|
| Sr. No | Ingredients | w/v |
| 1. | Loperamide | 11.2 |
| 2. | Ethanol | 76.7 |
| 3. | HPMC | 4.1 |
| 4. | Sodium lauryl sulphate | 4.0 |
| 5. | Yellow iron oxide | 1.5 |
| 6. | Banana Flavor | 2.5 |

1.4 Preparation of Levocetrizine Printing Ink—(Ink D)

TABLE 4

| | | Amount in Percent |
|---|---|---|
| Sr. No | Ingredients | w/w |
| 1 | Levocetirizine | 8 |
| 2 | BHA | 1 |
| 3 | BHT | 1 |
| 4 | Ethanol | 86 |
| 5 | PEG-6000 | 4 |

1.5 Preparation of Diclofenac Printing Ink—(Ink E)

TABLE 5

| | | Amount in Percent |
|---|---|---|
| Sr. No | Ingredients | w/w |
| 1 | Diclofenac | 8.0 |
| 2 | Ethanol | 86.83 |
| 3 | Na CMC | 4.3 |
| 4 | Neotame | 0.87 |

1.6 Preparation of Nifedipine Containing Ink—(Ink F)

TABLE 6

| | | Amount in Percent |
|---|---|---|
| Sr. No | Ingredients | w/w |
| 1 | Nifedipine | 7.0 |
| 2 | BHA | 1.5 |
| 3 | BHT | 1.5 |
| 4 | Ethanol | 86.5 |
| 5 | Gelatin | 2.0 |
| 6 | Potassium cholride | 1.5 |

1.7 Preparation of Vitamin D3 Ink—(Ink G)

TABLE 7

| | | Amount in Percent |
|---|---|---|
| Sr. No | Ingredients | w/v |
| 1 | Vitamin D3 | 4.30 |
| 2 | BHA | 3.48 |
| 3 | BHT | 3.48 |
| 4 | Ethanol | 81.0 |
| 5 | Ethyl cellulose | 1.74 |
| 6 | Sodium chloride | 1.0 |
| 7 | Xylitol | 5.0 |

### Example 2

Method of Making Ink of this Invention

Ink formulations given in example 1.1 to 1.7 were prepared by mixing the ingredients in solvent using a sonicator. Weighed quantities of Active Ingredient and excipients were added to the glass vial. To this accurately measured quantity of solvent ethanol was added and sonicated for about 10-20 minutes after capping the vial tightly. The ink thus prepared was stored under ambient conditions and used within 48 h for printing on placebo substrate.

### Example 3

Evaluation of Printing Ink

Ink formulations given in example 1.1 to 1.7 were evaluated for viscosity, density, surface tension, contact angle, Active Ingredient content etc. All the ink formulations possessed viscosity in the range of 8-12 mPa s, required for suitable printing. Surface tension and the density of the prepared ink compositions were in the range 22-30 mN per m and 1-1.5 kg/1 respectively. These ranges are required for continuous drop formation and fast drying of the ink. The prepared ink compositions were stable under stress conditions

Measurement of Viscosity

Viscosity was determined using an Ostwald U-tube viscometer. The time required for the solution to pass between two marks as it flowed under gravity through the vertical capillary of the viscometer was determined, and viscosities were calculated with reference to data for distilled water. All measurements were performed with the viscometer mounted in a thermostatted water bath (25±0.5° C.). Three replicates were performed for each solution and the results are presented as mean±standard deviation.

Measurement of Density

Clean and dry 50 ml volumetric flask was taken. To it 50 ml of ink was added and weight of the ink was taken using analytical balance. Density of the ink was determined as weight of the ink (Mass) divided by volume of the ink.

Measurement of Surface Tension

Surface tension of the ink formulations was measured using stalagmometer. The ink was filled in the stalagmometer and the drops were allowed to fall in clean and dry glass beaker. The drop falls from the stalagmometer when the weight (mg) is equal to the circumference (2πr) multiplied by the surface tension (σ). The surface tension can be calculated provided the radius of the tube (r) and mass of the fluid droplet (m) are known. Alternatively, since the surface tension is proportional to the weight of the drop, the fluid of interest may be compared to a reference fluid of known surface tension (typically water):

$$\frac{{m}_{1}}{{σ}_{1}} = \frac{{m}_{2}}{{σ}_{2}}$$

In the equation, m₁ and σ₁ represent the mass and surface tension of the reference fluid and m₂ and σ₂ the mass and surface tension of the fluid of interest. If we take water as a reference fluid,

$$σ = {σ}_{{H}_{2} O} \times \frac{m}{{m}_{{H}_{2} O}}$$

(surface tension=72.8 mN per m 20° C.) Data are presented as mean±standard deviation.

Measurement of Contact Angle

Contact angle is a measure of adherence between ink and the substrate. Contact angle of the ink was measured by dropping ink on the substrate at 90 degrees with the help of a micropipette and the angle formed by the drop was observed with the help of magnifying glass using protractor. Contact angle values indicate good adherence between ink and substrate. Lower the value better the adherence.

TABLE NO. 8
Properties of Active ingredient-loaded Ink compositions

| | | | Surface | |
|---|---|---|---|---|
| | Viscosity | Density | tension | Contact |
| Ink | (mPa s) | (kg/l) | (mN per m) | angle |
| Ink A | 10.1 ± 1.8 | 1.1 ± 0.9 | 30 ± 1.1 | 29.1° ± 1.8°. |
| Ink B | 12.1 ± 1.1 | 1.4 ± 1.2 | 22 ± 1.1 | 25.13° ± 1.0° |
| Ink C | 10.4 ± 1.8 | 1.2 ± 0.99 | 26 ± 0.94 | 25.1° ± 1.1° |
| Ink D | 8.0 ± 1.2 | 1.6 ± 0.12 | 26 ± 0.88 | 28.6° ± 1.1° |
| Ink E | 8.7 ± 1.9 | 1.3 ± 0.23 | 31 ± 0.65 | 22.1° ± 1.8° |
| Ink F | 10.2 ± 0.8 | 1.1 ± 0.91 | 26.5 ± 0.34 | 27.1° ± 0.8° |
| Ink G | 10.7 ± 0.56 | 1.1 ± 0.11 | 28 ± 1.2 | 28.3° ± 1.2° |

### Example 4

4. Method of Preparation of Preparation of Placebo Substrate

4.1 Preparation of Orally Dissolving Placebo Substrate (ODS) (Substrate 1)

TABLE 9

| | | Amount in Percent |
|---|---|---|
| Sr. No | Ingredients | w/w |
| 1 | HPMC | 71.1 |
| 2 | Propylene Glycol | 8.80 |
| 3 | Glycerin | 5.56 |
| 4 | Sucralose | 4.44 |
| 5 | Titanium dioxide | 2.1 |
| 6 | Mentha oil | 1.56 |
| 7 | Vitamin E Dry Powder 50% | 5.33 |
| 8 | Simethicone | 1.11 |

In a Stainless Steel container, weighed quantity of HPMC, propylene glycol, glycerin, Mentha oil, Simethicone, sucralose, titanium dioxide and purified water was added and stirred for 30 min using lab stirrer at 3500 rpm for homogeneous dispersion preparation. The dispersion was then casted using film casting machine in uniform thickness and dried at 80° C. for 10 minutes.

4.2 Preparation of Placebo Substrate (ODS) (Substrate 2)

TABLE 10

| | | Amount in Percent |
|---|---|---|
| Sr. No | Ingredients | w/w |
| 1 | HPMC | 80.5 |
| 2 | Propylene Glycol | 4.29 |
| 3 | Glycerin | 4.09 |
| 4 | Sucralose | 2.14 |
| 5 | Titanium dioxide | 1.41 |
| 6 | Mentha oil | 1.07 |
| 7 | Vitamin E Dry Powder 50% | 0.70 |
| 8 | Simethicone | 1.40 |
| 9 | Microcrystalline Cellulose (MCC) | 4.4 |

In a Stainless Steel container, weighted quantity of HPMC, propylene glycol, glycerin, mentha oil, Simethicone, MCC, Titanium dioxide, sucralose and purified water was added and stirred for 30 min using lab stirrer at 3500 rpm. for homogeneous dispersion preparation. The dispersion was then casted using film casting machine in uniform thickness and dried at 80° C. for 10 minutes.

4.3 Preparation of Placebo Substrate (ODS) (Substrate 3)

TABLE 11

| | | Amount in Percent |
|---|---|---|
| Sr. No | Ingredients | w/w |
| 1 | HPMC | 78.1 |
| 2 | Propylene Glycol | 4.29 |
| 3 | Glycerin | 4.29 |
| 4 | Sucralose | 2.14 |
| 5 | Titanium dioxide | 1.71 |
| 6 | Mentha oil | 1.07 |
| 7 | Vitamin E Dry Powder 50% | 0.70 |
| 8 | Simethicone | 1.20 |
| 9 | Gelatin | 6.5 |

In a Stainless Steel container, weighted quantity of HPMC, Gelatin, titanium dioxide, sucralose propylene glycol, glycerin, mentha oil, Simethicone and purified water was added and stirred for 30 min using lab stirrer at 3500 rpm for homogeneous dispersion preparation. The dispersion was then casted using film casting machine in uniform thickness and dried at 80° C. for 10 minutes.

4.4 Preparation of Placebo Substrate (ODS) (Substrate 4)

TABLE 12

| | | Amount in Percent |
|---|---|---|
| Sr. No | Ingredients | w/w |
| 1 | HPMC | 77.1 |
| 2 | Propylene Glycol | 4.29 |
| 3 | Glycerin | 4.29 |
| 4 | Sucralose | 2.14 |
| 5 | Titanium dioxide | 1.71 |
| 6 | Mentha oil | 1.07 |
| 7 | Vitamin E Dry Powder 50% | 0.71 |
| 8 | Simethicone | 1.43 |
| 9 | Polyethylene Glycol (PEG)-6000 | 7.8 |

In a Stainless Steel container, weighted quantity of HPMC, PEG-6000, propylene glycol, glycerin, Mentha oil, Simethicone, titanium dioxide, Sucralose and purified water was added and stirred for 30 min using lab stirrer at 3500 rpm for homogeneous dispersion preparation. The dispersion was then casted using film casting machine in uniform thickness and dried at 80° C. for 10 minutes.

4.5 Preparation of Placebo Substrate (Sublingual) (Substrate 5)

TABLE 13

| | | Amount in Percent |
|---|---|---|
| Sr. No | Ingredients | w/w |
| 1 | Sucralose | 3.33 |
| 2 | Sunset yellow colour | 0.11 |
| 3 | Orange flavor | 3.11 |
| 4 | HPMC | 61.11 |
| 5 | BHA | 2.22 |
| 6 | BHT | 1.11 |
| 7 | Glycerin | 7.79 |
| 8 | PEG 400 | 10.00 |
| 9 | Maltodextin | 11.22 |

In a Stainless Steel container, weighted quantity of HPMC, glycerin, PEG 400, maltodextrin, BHA, BHT, sucralose, orange flavour, sunset yellow colour and purified water was added and stirred for 30 min using lab stirrer at 3500 rpm for homogeneous dispersion preparation. The dispersion was then casted using film casting machine in uniform thickness and dried at 80° C. for 10 minutes.

4.6 Preparation of Placebo Substrate (Buccal) (Substrate 6)

TABLE 14

| | | Amount in Percent |
|---|---|---|
| Sr. No | Ingredients | w/w |
| 1 | Sucralose | 2.22 |
| 2 | HPMC K4M | 7.78 |
| 3 | Carbopol 974 | 5.56 |
| 4 | HPMC | 24.28 |
| 5 | HPC | 22.0 |
| 6 | Titanium dioxide | 3.30 |
| 7 | Glycerin | 4.47 |
| 8 | PEG 400 | 13.33 |
| 9 | Na CMC | 5.56 |
| 10 | Vitamin E acetate | 10.00 |
| 11 | Simethicone | 1.5 |

In a Stainless Steel container, weighted quantity of Sucralose, sodium CMC, HPMC K4M, Carbopol 974, glycerin, HPMC, HPC, PEG 400, Titanium dioxide, simethicone, Vitamin E acetate and purified water was added and stirred for 30 min using homogenizer at 3500 rpm. The dispersion was then casted using film casting machine in uniform thickness and dried at 80° C. for 10 minutes.

### Example 5

Printability Evaluation of Ink on Placebo Substrates

Active Ingredient loaded printing ink formulations prepared as per example no. 1.1-1.5 were used for printing on the prepared substrates as per example 3.1 to 3.6. Printing was done using pneumatic based extrusion technique. Active Ingredient printing using Ink composition as per example 1.6 and 1.7 was carried out by Continuous Inkjet printer. All the substrates were checked for cavity formation as well as adherence of ink on the substrate. The substrate forming cavity after dropping of the ink on its surface was referred as having poor printability, whereas the substrate not showing any cavity formation and good adherence of ink without spreading and fast drying of ink was referred as having Good printability.

TABLE 15
Preliminary Printability evaluation-

| INK | Printing on substrate (Placebo) | | | | | |
|---|---|---|---|---|---|---|
| Composition | Substrate 1 | Substrate 2 | Substrate 3 | Substrate 4 | Substrate 5 | Substrate 6 |
| Ink A | x | ✓ | ✓ | ✓ | ✓ | ✓ |
| Ink B | x | ✓ | ✓ | ✓ | ✓ | ✓ |
| Ink C | x | ✓ | ✓ | ✓ | ✓ | ✓ |
| Ink D | x | ✓ | ✓ | ✓ | ✓ | ✓ |
| Ink E | x | ✓ | ✓ | ✓ | ✓ | ✓ |
| Ink F | x | ✓ | ✓ | ✓ | ✓ | ✓ |
| Ink G | x | ✓ | ✓ | ✓ | ✓ | ✓ |

✓-Good printability,
x-Poor printability.

It was observed that substrate of example 4.1 was used for Active Ingredient printing by using Pneumatic based extrusion technique and Continuous InkJet printer. The cavity formation was observed immediately after printing. The printed substrate lost its mechanical strength and foldability. Therefore, modifications in the composition of substrate were made with the view to impart paper like qualities to the substrate. Composition of modified substrate is given in example 4.2, 4.3, 4.4, 4.5 and 4.6. Since printing of substrate 1 was not successful with all the inks, therefore, it was not evaluated after printing.

5. Evaluation of Physical Parameters of Substrate (Film) Before and After Printing—

5.1. Evaluation of Physical Parameters of Placebo Substrate Before Printing—

After preliminarily evaluating printability of all the placebo film substrates, physicochemical parameters of substrates were compared before printing and after printing. As the required critical attributes for the oral dissolving strip (ODS) (Substrate 2 to 4) and sublingual films (substrate 5) were folding endurance, fast disintegration and mechanical strength, these attributes were evaluated after Active Ingredient printing also (Table 5.2) to ensure the quality of Active Ingredient printed films.

Measurement of Folding Endurance—

The folding endurance along with tensile strength of a film is related to the flexibility of a film and hence represents its physical stability during manufacturing, packing and use. It was measured manually by firmly folding a film repeatedly through the middle. The number of folds on the same crease, required to produce crack in the film was noted as the value of folding endurance. It was measured by repeatedly folding a film at the same point until it breaks. Folding endurance value is number of times the film is folded without breaking. A film of 5 cm2 was taken and folded it repeatedly at 180° in both the side until it breaks Minimum acceptable limit for folding endurance is 20-25 times it should be folded without breaking.

Disintegration Time

Disintegration test was performed in the USP disintegration apparatus. Simulated salivary fluid (pH 6.8) was used as the medium. The films were placed in the tubes of the container and the discs were placed over it. The average disintegration time of six films from each formulation was noted. The minimum acceptable limit for disintegration of the oral thin film is 30 sec.

Tensile Strength

Tensile strength is maximum stress applied to a point at which the strip specimen breaks (Felton et al., 2008). Film strip of dimension 2×2 cm2 and free from air bubbles or physical imperfections was held between two clamps positioned at a distance of 3 cm apart. A cardboard was attached on the surface of the clamp via a double sided tape to prevent the film from being cut by the grooves of the clamp. During measurement, the strips were pulled at the bottom clamp by adding weights in pan till the film breaks. The force was measured when the films broke.

Tensile strength (kg/mm2)=Force at Break/Initial cross sectional area of the film (mm²).

The average of tensile strength of 3 films was taken as final reading.

Substrate 6 (Buccal film) was shown to have longer disintegration time before and after printing since the required attribute for buccal films was adherence to buccal mucosa and slow disintegration of the substrate.

TABLE 16
Evaluation of physical parameters of substrate before printing

| | Substrate Type (sub) | | | | | |
|---|---|---|---|---|---|---|
| Properties | Sub 1 | Sub 2 | Sub 3 | Sub 4 | Sub 5 | Sub 6 |
| Folding | 10 | 9 | 11 | 08 | 09 | 27 |
| endurance | | | | | | |
| Disintegration | 8 | 11 | 11 | 11 | 12 | 30 |
| (sec) | | | | | | minutes |
| Tensile | 12 | 11 | 14 | 12 | 11 | 24 |
| strength | | | | | | |
| (kg/mm2) | | | | | | |

5.2—Evaluation of Physicochemical Parameters of Placebo Substrate after Printing

For folding endurance, disintegration time, and tensile strength methods given above were used and for the analysis of drug content for assay determination and during stability studies at 40° C./75% RH, respective monographs in pharmacopoeia were referred.

TABLE 17
Evaluation of physical parameters of substrate after printing-

| | | Ink A | Ink B | Ink C | Ink D | Ink E | Ink F | Ink G |
|---|---|---|---|---|---|---|---|---|
| Folding | Sub 2 | 11 | 10 | 08 | 12 | 10 | 09 | 11 |
| endurance | Sub 3 | 11 | 07 | 10 | 12 | 07 | 13 | 12 |
| | Sub 4 | 08 | 07 | 11 | 11 | 07 | 09 | 12 |
| | Sub 5 | 09 | 08 | 10 | 12 | 10 | 12 | 09 |
| | Sub 6 | 27 | 25 | 28 | 25 | 22 | 27 | 28 |
| Disintegration | Sub 2 | 12 | 11 | 11 | 12 | 14 | 14 | 12 |
| (sec) | Sub 3 | 12 | 11 | 14 | 10 | 11 | 08 | 12 |
| | Sub 4 | 12 | 12 | 11 | 10 | 14 | 11 | 14 |
| | Sub 5 | 11 | 11 | 10 | 14 | 12 | 13 | 11 |
| | Sub 6 | 30 min | 32 min | 30 min | 32 min | 31 min | 32 min | 34 min |
| Tensile | Sub 2 | 11 | 09 | 09 | 11 | 12 | 09 | 12 |
| strength | Sub 3 | 14 | 11 | 12 | 11 | 11 | 12 | 14 |
| (kg/mm2) | Sub 4 | 11 | 11 | 12 | 11 | 10 | 12 | 11 |
| | Sub 5 | 11 | 10 | 12 | 11 | 14 | 12 | 12 |
| | Sub 6 | 24 | 25 | 26 | 26 | 21 | 23 | 24 |
| Assay (%) | Sub 2 | 99.20 | 99.52 | 98.71 | 99.18 | 99.42 | 99.11 | 98.68 |
| | Sub 3 | 99.23 | 99.51 | 98.72 | 99.10 | 99.44 | 99.13 | 98.47 |
| | Sub 4 | 99.29 | 99.56 | 98.70 | 99.10 | 99.47 | 99.18 | 99.45 |
| | Sub 5 | 98.92 | 99.51 | 98.79 | 99.01 | 99.24 | 99.14 | 99.17 |
| | Sub 6 | 98.72 | 99.55 | 98.87 | 99.17 | 99.43 | 99.12 | 99.46 |
| Percent drug | Sub 2 | 95.26 | 96.55 | 96.44 | 96.32 | 95.44 | 92.34 | 94.33 |
| content after 6 | Sub 3 | 96.34 | 96.15 | 95.27 | 96.55 | 94.66 | 97.22 | 94.52 |
| months of | Sub 4 | 96.22 | 94.15 | 96.42 | 94.45 | 95.46 | 96.32 | 97.74 |
| storage at 40° | Sub 5 | 94.12 | 94.22 | 94.54 | 98.46 | 94.15 | 97.24 | 97.54 |
| C./75% RH | Sub 6 | 95.12 | 94.55 | 97.04 | 95.48 | 96.54 | 96.17 | 97.22 |

### REFERENCES

1. Janssen, E. M., Schliephacke, R., Breitenbach, A., Breitkreutz, J., 2013. Drug printing by flexographic printing technology—a new manufacturing process for orodispersible films. Int. J. Pharm. 441, 818-825.
2. Melendez, P. A., Kane, K. M., Ashvar, C. S., Albrecht, M., Smith, P. A., 2008. Thermal inkjet application in the preparation of oral dosage forms: dispensing of prednisolone solutions and polymorphic characterization by solidstate spectroscopic techniques. J. Pharm. Sci. 97, 2619-2636.
3. Genina, N., Janssen, E. M., Breitenbach, A., Breitkreutz, J., Sandler, N., 2013b. Evaluation of different substrates for inkjet printing of rasagiline mesylate. Eur. J. Pharm. Biopharm. 85, 1075-1083.
4. Sandler, N., Maattanen, A., Ihalainen, P., Kronberg, L., Meierjohann, A., Viitala, T., Peltonen, J., 2011. Inkjet printing of drug substances and use of porous substrates—towards individualized dosing. J. Pharm. Sci. 100, 3386-3395.
5. Raijada, D., Genina, N., Fors, D., Wisaeus, E., Peltonen, J., Rantanen, J., Sandler, N., 2013. A step toward development of printable dosage forms for poorly soluble drugs. J. Pharm. Sci. 102, 3694-3704.
6. Vuddanda P R, Alomari M, Dodoo C C, Trenfield J, Velaga S, Basit A W, Gaisford S; 2018. Personalisation of warfarin therapy using thermal ink-jet printing. European Journal of Pharmaceutical Sciences. Eur. J. Pharm. Biopharm. 117, 80-87
7. A. B. M. Buanz, M. H. Saunders, A. W. Basit, S. Gaisford, Preparation of personalized-dose salbutamol sulphate oral films with thermal ink-jet printing, Pharmaceutical Research 28 (2011) 2386-2392
8. A. B. M. Buanz, et al., Ink-jet printing versus solvent casting to prepare oral films: Effect on mechanical properties and physical stability, Int J Pharmaceut (2014)
8. C. Planchette, et al., Printing medicines as orodispersible dosage forms: Effect of substrate on the printed micro-structure, Int J Pharmaceut (2015). U.S. Pat. No. 6,207,258 B1 composition and method for improved ink jet printing performance.

1. An orodispersible thin film for providing a substrate for printing of at least one active ingredient on its surface that remains free from cavities after printing.

2. The orodispersible thin film according to claim 1 characterized by having folding endurance of minimum 8 times without breaking, disintegration time of minimum 10 seconds after coming in contact with water and minimum mechanical/tensile strength of 9 kg/mm².

3. The orodispersible thin film according to claim 1 wherein the film is one selected from the group consisting of buccal, sub-lingual, oral, vaginal, rectal and ocular film.

4. The orodispersible thin film according to claim 1, wherein the film:
a. is made from one or more film forming polymers selected from group consisting of HPMC, HPC, Polyethylene Oxide, Pullulan, maltodextrin, sodium alginate, HPMC, Gelatin, carrageenan, chitosan, Sodium CMC, Polyvinyl Pyrrolidone, Polyvinyl Alcohol, and Modified starch; and
b. comprises one or more plasticizers selected from group consisting of Polyethylene Glycol, propylene glycol, glycerin, sorbitol, triacetin, and mannitol.

5. A substrate for making an orodispersible thin film that has printing on its surface with at least one Active Ingredient for oral/transmucosal delivery.

6. The substrate according to claim 5 characterized in that the said substrate has paper like surface roughness, porosity and absorptivity for the purpose of printing; and foldability and mechanical strength for the purpose of withstanding handling in course of its manufacturing, packaging, storage, transportation and use.

7. The substrate according to claim 6 comprising of one or more of ingredient/s that act/s as adsorbent/s and impart/s required roughness to the surface of the substrate.

8. The substrate according to claim 6 when the said ingredient is selected from the group consisting of MCC, gelatin, Polyethylene Glycol-2000, Polyethylene Glycol-4000, Polyethylene Glycol-6000, Titanium Dioxide, Kaolin, Kieselguhr, Bentonite and Neucilin.

9. The substrate according to claim 6 wherein the selected ingredient is used in the range 2-10% w/w.

10. A method for making a substrate for making an orodispersible thin film for providing a substrate for printing of at least one Active ingredient on its surface that remains free from cavities after printing.

11. The method for making the substrate according to claim 10 comprising the steps of:
a. adding weighted quantity of film forming polymer, propylene glycol, glycerin, mentha oil, Simethicone and purified water was added in a Stainless Steel container, stirring to get a mixture;
b. adding one or a mixture of polymers that would provide paper like consistency to the mixture upon drying for the purpose of printing of the Active Ingredient, and homogenized into a homogenous dispersion;
c. making the mixture into a homogeneous dispersion;
d. casting the homogeneous dispersion in uniform thickness, and
e. drying to get a film.

12. The method for making the substrate according to claim 11 wherein:
a. the polymer is selected from a group consisting of HPMC, HPC, Polyethylene Oxide, Pullulan, maltodextrin, sodium alginate, HPMC, Gelatin, carrageenan, chitosan, Sodium CMC, Polyvinyl Pyrrolidone, Polyvinyl Alcohol, and Modified starch;
b. Casting of the film is done using film casting machine; and
c. drying is done at 80° C. for 10 minutes.

13. The method for making the substrate according to claim 10 wherein the substrate is mucoadhesive.

14. An ink for printing on an orodispersible film at least one Active ingredient without causing cavities after printing.

15. The ink according to claim 14 characterized by the fact that the ink does not cause dissolution of the substrate during printing.

16. The ink according to claim 14,
a. that is edible;
b. that dries fast after printing on a substrate;
c. that comprises surface tension, viscosity and contact angle required for adherence of ink to the substrate;
d. that does not crystallize on drying;
e. that remains stable at least for six months at 40° C. and 75% RH;
f. that comprises one or more viscosity modifiers comprising cellulosic polymers selected from the group consisting of HPC, ethyl cellulose, sodium alginate, HPMC, chitosan, xanthan gum, pectin, carrageenan and antioxidants like Butylated Hydroxy Anisole, Butylated Hydroxy Toluene, Tocopherol, Sodium bisulfite, sodium metabisulfite, and conductivity imparter like sodium chloride or other salts appropriately equivalent to Sodium Chloride for the purpose of the ink composition;
g. that comprises, one or more surfactant/s to solubilise the Active Ingredient, the surfactants being one or more selected from the group consisting of Tween 80, Tween 20, Span 80, Span 20, Brij, Poly Ethylene Glycols, pluronics, sorbitol esters, alkyl sulphates, alkyl ether sulphate, polyfatty acid esters, polyoxyethylene glycol fatty acid ester, polyoxyethylene stearate and Vitamin E acetate;
h. that comprises excipients that are pharmaceutically and/or nutraceutically acceptable;
i. that comprises one or more Active Ingredient selected from the group consisting of a Active Ingredient selected from the category of BCS Class I and II or other pharmaceutically effective ingredient or a nutraceutical ingredient;
j. that remains stable for at least six months at 40° C./75% RH;
k. that comprises ethanol or hydroalcoholic mixture as a solvent as well as antioxidants like Butylated Hydroxy Toluene, Butylated Hydroxy Anisole, Tocopherol, surfactants, and viscosity enhancers like cellulosic polymers selected from the group consisting of HPC, ethyl cellulose, sodium alginate, Hydroxymethyl cellulose of Low viscosity;
l. that dries fast when printed on the substrate; and
m. that is printable on the substrate using a continuous Inkjet printer or pneumatic based extrusion printer.
